(19) United States
(12) Plant Patent Application Publication
(10) Pub. No.: US 2003/0084495 P1
(43) Pub. Date: May 1, 2003

(54) GYPSOPHILA PLANT NAMED 'DANGYPFUN'

(76) Inventor: Gabriel Danziger, Nir-Zvi (IL)

Correspondence Address: FOLEY AND LARDNER, SUITE 500, 3000 K STREET NW, WASHINGTON, DC, 20007, US

(21) Appl. No.: 09/984,618

(22) Filed: Oct. 30, 2001

(51) Int. Cl. A01H 5/00

(52) U.S. Cl. PLT/354

(57) ABSTRACT

A new and distinct Gypsophila plant named ‘Dangypfun’ characterized by having semi double, white flowers, very small, 4-5 mm, 1500-2000 flowers per stem; buds remain unfolded for several days; flowers arranged in clusters, maintain stablility during hot season, 80-90 cm in height, open formation; apical dominance growth; 10-14 days cut flower longevity.

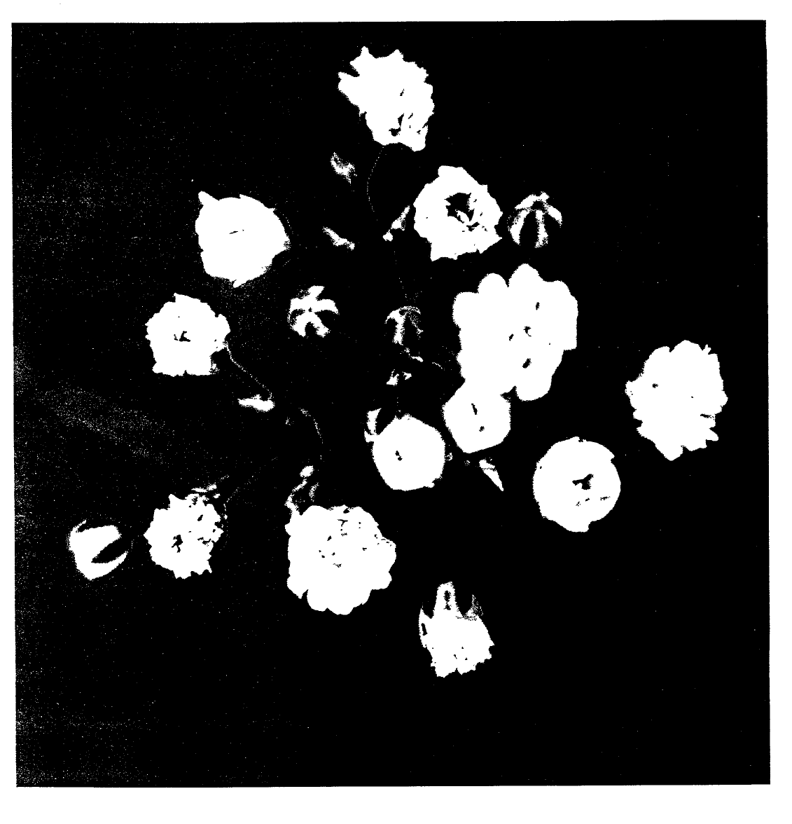

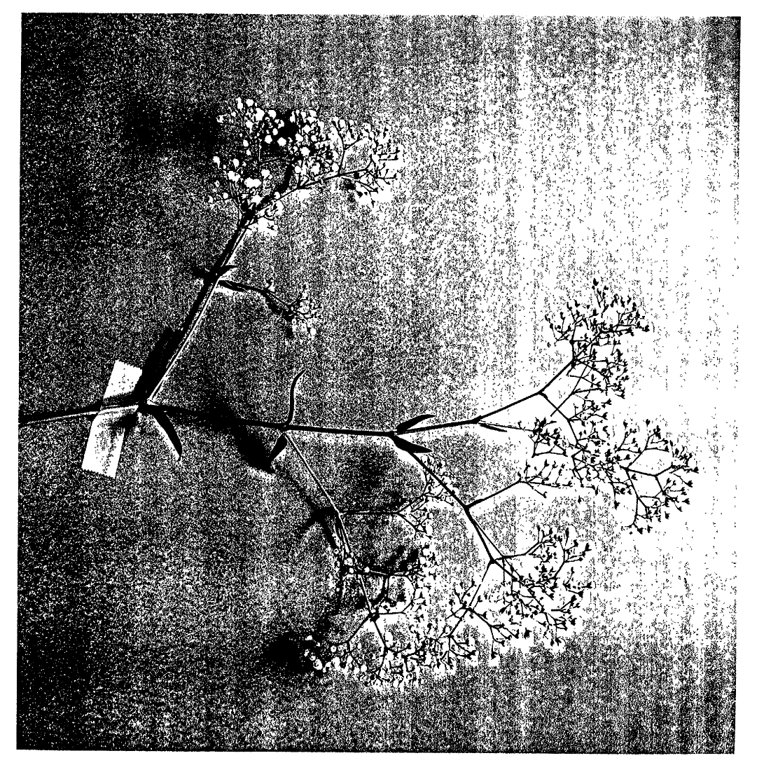

### BACKGROUND OF THE INVENTION

The present invention relates to a new and distinct cultivar of Gypsophila plant, botanically known as Gypsophila paniculata, hereinafter referred to by the cultivar name ‘Dangypfun’.

‘Dangypfun’ is a spontaneous mutant of cultivar Gypsophila ‘Dangypmini’ selected in a cultivated environment in Moshav Mishmar Hashiva, Israel by the inventor, Gabriel Danziger. Asexual reproduction of the new cultivar by side shoot cutting was first performed on January, 2001, in Moshav Mishmar Hashiva, Israel and has demonstrated that the combination of characteristics as herein disclosed for the new cultivar are firmly fixed, reproduce true to type, and are retained through successive generations of asexual reproduction.

### BRIEF DESCRIPTION OF THE INVENTION

The following traits have been repeatedly observed and are determined to be basic characteristics of ‘Dangypfun’ which in combination distinguish this Gypsophila as a new and distinct cultivar:

1. semi double, white flowers, very small, 4-5 mm, 1500-2000 flowers per stem;

2. buds remain unfolded for several days;

3. flowers arranged in clusters, maintain stablility during hot season, plant height of 80-90 cm, open formation;

4. apical dominance growth habit;

5. 10-14 days cut flower longevity.

‘Dangypfun’ has not been observed under all possible environmental conditions. The phenotype of the new cultivar may vary significantly with variations in environment such as temperature, light intensity, and day length without any change in the genotype of the plant. The following observations, measurements and values describe the new cultivar as grown in Moshav Mishmar Hashiva, Israel under conditions which closely approximate those generally used in commercial practice.

Of the many commercial cultivars known to the present inventor, the most similar in comparison to ‘Dangypfun’ is the cultivar ‘Dangypmini’. In comparison to ‘Dangypmini’, flowers of ‘Dangypfun’ are smaller, have less petals during low light intensities, and the fully developed buds of ‘Dangypfun’ remain unfolded for several days.

### BRIEF DESCRIPTION OF THE DRAWING

The accompanying photographic drawings show typical flower and foliage characteristics of ‘Dangypfun’ with colors being as true as possible will illustrations of this type. The first drawing is a close-up view of a flowering stem segment, and the second drawing shows a flowering stem of the instant plant.

### DETAILED BOTANICAL DESCRIPTION

The following observations, measurements and values describe the new cultivar as grown in Moshav Mishmar hashiva, Israel under conditions which closely approximate those generally used in commercial practice recommended for Gypsophila plants, in open field during summer and in a protected greenhouse during winter. The minimum temperature during winter is 15° C., the summer temperature ranging from 18-40° C. Color references are made to The Royal Horticultural Society Colour Chart (RHS), except where general colors of ordinary significance are used.

Origin: Spontaneous mutation.

Parentage:

Male parent.—None.

Female parent.—Gypsophila ‘Dangypmini’ Spontaneous mutation arose in commercial ‘Dangypmini’ plant.

Classification:

Botanical.—Hybrid of Gypsophila paniculata.

Commercial.—Gypsophila cv. Dangypfun.

Species designation.—None.

Propagation: Side shoot cuttings

Plant:

General appearance and form.—Height: 80-90 cm Width: 70-80 cm Habit: Apical dominance Form: Bush upright.

Flowering response.—During long day conditions.

Flowering season.—Spring, summer and fall, in winter only with supplemental lighting.

Vigor/Growth rate.—In summer flowers 9-10 weeks after planting, in winter flowers 13-14 weeks after planting.

Winter hardiness.—When grown in an open field, moderate winter weather is withstood.

Lastingness of the individual bloom.—10-14 days.

Rooting.—Application of rooting hormone, kept wet, direct sunlight avoided.

Fragrance.—Typical for Gypsophila.

Foliage:

Overall shape of leaf.—Base: Truncate Tip: Acuminate.

Margin.—Entire.

Texture.—Rough.

Main color of upper surface.—Mature leaf: Green 139 A Immature leaf: Green 139 A.

Main color of lower surface.—Mature leaf: Green 137 A Immature leaf: Green 137 A.

Venation color.—None.

Size.—Length: 6-7 cm Width: 10-12 mm.

Leaf base shape.—Truncate.

Leaf apex shape.—Acuminate.

Stipules.—None.

Inflorescence:

Natural flowering season.—Spring, summer and fall, during long day conditions.

Corolla.—Form: Ball shaped Shape: Ball shaped Average Number: 1500-2000 Size: 4-5 mm in diameter Petal Number: 44-45 on a long day with high light intensity 25-30 on a short day with low light intensity Petal Shape: Lanceolate with emarginate tip Petal Markings: None Petal Color: Upper surface: White 155 D Lower surface: White 155 D.

Stem:

Quanity.—6-8 stems per plant.

Average length.—80-90 cm.

Average diameter.—4-5 mm.

Color.—Green 143 B.

Internode length.—4-7 cm.

Bud:

Response.—Long day conditions.

Color.—White 155 D.

Size before opening.—2 mm.

Pedicel length.—4-6 mm.

Pedicel color.—Green 143 B.

Reproductive Organs: None

Disease Resistance: Not susceptible to most pests and diseases under regular growing conditions.

I claim:

1. A new and distinct Gypsophila plant named ‘Dangypfun’, substantially as illustrated and described herein.
